(19) United States
(12) Patent Application Publication
(10) Pub. No.: US 2025/0248876 A1
(43) Pub. Date: Aug. 7, 2025

(54) TRUNK SUPPORT EXOSKELETON WITH ONE POWERED ACTUATOR

(71) Applicants: The Regents of the University of California, Oakland, CA (US); suitX, Inc., Emeryville, CA (US)

(72) Inventors: Wayne TUNG, Emeryville, CA (US); Homayoon KAZEROONI, Oakland, CA (US); Jing-Song HUANG, Emeryville, CA (US); YI ZENG, Emeryville, CA (US); Zhendong LIU, Emeryville, CA (US)

(73) Assignees: The Regents of the University of California, Oakland, CA (US); suitX, Inc., Emeryville, CA (US)

(21) Appl. No.: 18/854,714

(22) PCT Filed: Apr. 11, 2023
(86) PCT No.: PCT/US2023/018217

§ 371 Date: Oct. 7, 2024

Related U.S. Application Data
(60) Provisional application No. 63/362,778, filed on Apr. 11, 2022.
(60) Provisional application No. 63/362,779, filed on Apr. 11, 2022.

(51) Int. Cl. A61H 1/02; A61H 3/00; B25J 9/00

(52) U.S. Cl. CPC A61H 1/0229; A61H 3/00; B25J 9/0006; A61H 2003/001; A61H 2003/007; A61H 2201/1207; A61H 2201/14; A61H 2201/1619; A61H 2201/1623; A61H 2201/164; A61H 2201/1652; A61H 2201/1669; A61H 2201/1671; A61H 2201/5064; A61H 2201/5069; A61H 2205/081

(57) ABSTRACT

Some embodiments described herein are directed to a trunk supporting exoskeleton for reducing muscle forces in a wearer's back during forward lumbar flexion. The trunk supporting exoskeleton can include a supporting trunk frame, a first thigh link, a second thigh link, an actuator, a shaft pulley, a housing pulley, a shaft line, and a housing line. The actuator can include an actuator housing and an actuator shaft. When the wearer is bent forward relative to a vertical gravitational line in a sagittal plane, the actuator can generate an actuator resistive torque between the actuator housing and the actuator shaft. The actuator resistive torque between the actuator housing and the actuator shaft can generate tensile forces in the housing line and the shaft line, thereby generating extension torques between the respective first and second thigh links and the supporting trunk frame.

### CROSS-REFERENCE TO RELATED APPLICATIONS

This application claims the benefit of U.S. Provisional Patent Application No. 63/362,778, filed Apr. 11, 2022, and U.S. Provisional Patent Application No. 63/362,779, filed Apr. 11, 2022. Each of these applications is incorporated herein by reference in its entirety.

International Application No. To Be Assigned (“AN ACTUATOR FOR AN EXOSKELETON,” Inventors Wayne TUNG et al., Attorney Docket No. 5085.004PC03), filed on the same day herewith, is incorporated herein in its entirety by reference thereto.

### TECHNICAL FIELD

The present disclosure relates generally to exoskeleton systems and more specifically to trunk support exoskeleton systems.

### BACKGROUND

It may be desirable to reduce forces in a wearer's back during lumbar flexion (e.g., during stooping or bending).

### SUMMARY

Some embodiments described herein are directed to a trunk supporting exoskeleton for reducing muscle forces in a wearer's back during forward lumbar flexion. The trunk supporting exoskeleton can include a supporting trunk frame, a first thigh link, a second thigh link, an actuator, a shaft pulley, a housing pulley, a shaft line, and a housing line. The supporting trunk frame can be configured to be coupled to the wearer's trunk. The first thigh link can be configured to be coupled to one of the wearer's thighs. The second thigh link can be configured to be coupled to another of the wearer's thighs. Each of the first and second thigh links can be rotatably coupled to the supporting trunk frame such that the respective first or second thigh links can flex or extend relative to the supporting trunk frame. The actuator can be coupled to the supporting trunk frame. The actuator can include an actuator housing and an actuator shaft. The actuator shaft and the actuator housing can be rotatable relative to the supporting trunk frame. The shaft pulley can be coupled to the actuator shaft. The housing pulley can be coupled to the actuator housing. The shaft line can have a first end wound onto the shaft pulley and a second end coupled to the first thigh link. The housing line can have a first end wound onto the housing pulley and a second end coupled to the second thigh link. When the wearer is bent forward relative to a vertical gravitational line in a sagittal plane, the actuator can generate an actuator resistive torque between the actuator housing and the actuator shaft. The actuator resistive torque between the actuator housing and the actuator shaft can generate tensile forces in the housing line and the shaft line, thereby generating extension torques between the respective first and second thigh links and the supporting trunk frame.

In some embodiments, the first thigh link can include a first thigh link pulley. In some embodiments, the second thigh link can include a second thigh link pulley. In some embodiments, the second end of the shaft line is wound onto the first thigh link pulley such that a tensile force in the shaft line provides an extension torque between the first thigh link and the supporting trunk frame. In some embodiments, the second end of the housing line is wound onto the second thigh pulley such that a tensile force in the housing line provides an extension torque between the second thigh link and the supporting trunk frame.

In some embodiments, when the wearer is not bent forward relative to the vertical gravitational line, the actuator does not produce an actuator resistive torque between the actuator housing and the actuator shaft.

In some embodiments, when the wearer is not bent forward relative to the vertical gravitational line, the actuator generates a substantially small actuator resistive torque between the actuator housing and the actuator shaft allowing for substantially free movement of the thigh links.

In some embodiments, when the wearer is not bent forward relative to the vertical gravitational line and the thigh links are in a reciprocating mode indicative of walking, the actuator generates a substantially small actuator resistive torque allowing for substantially free movement of the thigh links.

In some embodiments, the shaft line and the housing line each comprise an element or combination of elements selected from a group consisting of wire, cable, belts, fabric rope, plastic rope, cord, twine, chain, wire rope and string.

In some embodiments, the actuator comprises an element or combination of elements selected from a group consisting of AC (alternating current) motors, brush-type DC (direct current) motors, brushless DC motors, electronically commutated motors (ECMs), stepper motors, and combinations thereof.

In some embodiments, the actuator includes a transmission system.

In some embodiments, the transmission system includes an element or combination of elements selected from a group consisting of harmonic drives, planetary gears, ball screw mechanisms, lead screw mechanisms, worm gears and combinations thereof.

In some embodiments, the transmission system includes an element or combination of elements selected from a group consisting of gears, worm gears, gear trains, pulleys, lines, belts, toothed belts, toothed pulleys, planetary gears, harmonic drives, spur gears, flexible belts, wire ropes, ropes, ball screw mechanisms, and lead screw mechanisms.

In some embodiments, the trunk supporting exoskeleton includes a controller to send a signal to the actuator to generate the actuator resistive torque between the actuator housing and the actuator shaft when the wearer is bent forward relative to the vertical gravitational line.

In some embodiments, the actuator resistive torque is a function of how much the wearer is bent forward relative to the vertical gravitational line.

In some embodiments, the actuator resistive torque increases as an angle of the supporting trunk frame relative to the vertical gravitational line increases.

In some embodiments, the actuator resistive torque decreases as an angle of the supporting trunk frame relative to the vertical gravitational line decreases.

In some embodiments, the actuator resistive torque is a function of an angular velocity of the supporting trunk frame in the sagittal plane.

In some embodiments, the actuator resistive torque decreases as a forward angular velocity of the supporting trunk frame in the sagittal plane increases.

In some embodiments, the actuator resistive torque increases as a forward angular velocity of the supporting trunk frame in the sagittal plane decreases.

In some embodiments, the actuator resistive torque decreases as a backward angular velocity of the supporting trunk frame in the sagittal plane increases.

In some embodiments, the actuator resistive torque increases as a backward angular velocity of the supporting trunk frame in the sagittal plane decreases.

In some embodiments, the controller sends a signal to the actuator to generate a substantially small actuator resistive torque between the actuator housing and the actuator shaft when wearer is not bent forward relative to the vertical gravitational line.

In some embodiments, the trunk supporting exoskeleton includes a tilt sensor and a controller. In some embodiments, the tilt sensor generates a tilt signal indicative of an angle of the supporting trunk frame relative to the vertical gravitational line in the sagittal plane. In some embodiments, the controller sends a signal to the actuator to generate the actuator resistive torque between the actuator housing and the actuator shaft when the tilt signal indicates an angle of the supporting trunk frame relative to the vertical gravitational line that is greater than a predetermined angle.

In some embodiments, the tilt sensor includes an element or combination of elements selected from a group consisting of Inertial Measurement Units (IMU), inclinometers, encoders, and angle sensors.

In some embodiments, the actuator resistive torque is a function of the tilt signal.

In some embodiments, the controller sends a signal to the actuator to generate a substantially small actuator resistive torque between the actuator housing and the actuator shaft when the tilt signal indicates that the wearer is not bent forward relative to the vertical gravitational line.

In some embodiments, the trunk supporting exoskeleton includes a shaft line jacket enclosing the shaft line. In some embodiments, the shaft line jacket is secured to the supporting trunk frame to facilitate a size adjustment of the supporting trunk frame without adjustment to the size of the shaft line.

In some embodiments, the trunk supporting exoskeleton includes a housing line jacket enclosing the housing line. In some embodiments, the housing line jacket is secured to the supporting trunk frame to facilitate a size adjustment of the supporting trunk frame without adjustment to the size of the housing line.

In some embodiments, the actuator generates the actuator resistive torque by use of electric power.

In some embodiments, the actuator includes an actuator spring. In some embodiments, a first end of the actuator spring is coupled to the actuator shaft. In some embodiments, a second end of the actuator spring is free in a first range of rotation of the actuator shaft relative to the actuator housing. In some embodiments, a second end of the actuator spring is constrained by the actuator housing in a second range of rotation of the actuator shaft relative to the actuator housing. In some embodiments, in the first range of rotation, the actuator generates the actuator resistive torque by use of electric power. In some embodiments, in the second range of rotation, the spring generates at least part of the actuator resistive torque.

In some embodiments, the actuator spring comprises an element or combination of elements selected from a group consisting of coil springs, leaf springs, bungee cords, rotary springs, elastomer cords, elastic cords, fabric cords, plastic cords, cords, twine, wire rope elastomers, and string.

In some embodiments, in a first range of rotation of the actuator shaft relative to the actuator housing the actuator generates the actuator resistive torque by use of electric power. In some embodiments, in a second range of rotation of the actuator shaft relative to the actuator housing, the actuator resistive torque is the addition of a torque generated by a spring and a torque generated by use of the electric power.

In some embodiments, the supporting trunk frame comprises a lower frame part configured to partially surround the wearer's trunk and coupled to the first and second thigh links at two sides of the wearer.

In some embodiments, the supporting trunk frame comprises a spine frame part coupled to the lower frame part.

In some embodiments, the spine frame part is adjustable in length to accommodate wearers of various heights.

In some embodiments, the lower frame part is adjustable in width to accommodate wearers of various width sizes.

In some embodiments, the lower frame part is adjustable in depth to accommodate wearers of various depth sizes.

In some embodiments, wherein the supporting trunk frame includes an upper frame part coupled to a spine frame part and configured to impose a supporting trunk force on the wearer's trunk and chest area.

In some embodiments, the upper frame part is configured to rotate relative to the spine frame part along an axis substantially parallel to the wearer's spine.

In some embodiments, the spine frame part is configured to rotate relative to a lower frame part along an axis substantially parallel to the wearer's spine.

In some embodiments, the upper frame part is configured to rotate relative to the spine frame part along an axis substantially parallel to one of the wearer's lumbar spine mediolateral flexion and extension axes.

Some embodiments described herein are directed to a trunk supporting exoskeleton for reducing muscle forces in a wearer's back during forward lumbar flexion. The trunk supporting exoskeleton can include a supporting trunk frame, a first thigh link, a second thigh link, and an actuator. The supporting trunk frame can be configured to be coupled to the wearer's trunk. The first thigh link can be configured to be coupled to one of the wearer's thighs. The second thigh link can be configured to be coupled to another of the wearer's thighs. Each of the first and second thigh links can be rotatably coupled to the supporting trunk frame such that the respective first or second thigh links can flex or extend relative to the supporting trunk frame. The actuator can be coupled to the supporting trunk frame. The actuator can include an actuator housing and an actuator shaft. The actuator can be free to rotate relative to the supporting trunk frame. The actuator shaft can be coupled to the first thigh link and the actuator housing can be coupled to the second thigh link. When the wearer bends forward in a sagittal plane, the actuator can generate an actuator resistive torque between the actuator housing and the actuator shaft, thereby generating extension torques between the respective first and second thigh links and the supporting trunk frame.

In some embodiments, the trunk supporting exoskeleton includes a shaft pulley, a housing pulley, a shaft line, and a housing line. In some embodiments, the shaft pulley is coupled to the actuator shaft. In some embodiments, the housing pulley is coupled to the actuator housing. In some embodiments, the shaft line has a first end wound onto the shaft pulley and a second end coupled to the first thigh link. In some embodiments, the housing line has a first end wound onto the housing pulley and a second end coupled to the second thigh link. In some embodiments, the actuator resistive torque can generate tensile forces in the housing line and the shaft line, thereby generating extension torques between the respective first and second thigh links and the supporting trunk frame.

### BRIEF DESCRIPTION OF THE DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows a perspective view of a person wearing a trunk support exoskeleton.

FIG. 2 shows a perspective view of the trunk support exoskeleton of FIG. 1.

FIG. 3 shows another perspective view of the trunk support exoskeleton of FIG. 1.

FIG. 4 shows a side view of a person wearing the trunk support exoskeleton of FIG. 1, with the person bent forward in the sagittal plane.

FIG. 5 shows a perspective view of an actuator with a housing pulley and a shaft pulley for the trunk support exoskeleton of FIG. 1.

FIG. 6 shows a cross-sectional view of the actuator of FIG. 5.

FIG. 7 shows a perspective view of the actuator of FIG. 5, with the shaft pulley removed for purposes of illustration.

FIG. 8 shows a cross-sectional view of the actuator of FIG. 5, with the shaft pulley removed for purposes of illustration.

FIG. 9 shows a perspective view of the actuator of FIG. 5, with the housing pulley and shaft pulley removed for purposes of illustration.

FIG. 10 shows a cross-sectional view of the actuator of FIG. 5, with the housing pulley and shaft pulley removed for purposes of illustration.

FIG. 11 shows an enlarged view of a portion of the trunk support exoskeleton of FIG. 1.

FIG. 12 shows an enlarged view of a portion of a trunk support exoskeleton showing an alternative connection between a shaft line and thigh link.

FIG. 13 is a schematic showing how a resistive torque between a housing and shaft of an actuator of the trunk support exoskeleton of FIG. 1 generates an extension torque between thigh links and a trunk support frame of the trunk support exoskeleton.

FIG. 14 shows a diagram of forces on a person's back when bending forward in the sagittal plane.

FIG. 15 shows a flow chart of a control algorithm for controlling a trunk support exoskeleton.

FIG. 16 shows an enlarged view of a portion of the trunk support exoskeleton of FIG. 1.

FIG. 17 shows an enlarged view of a portion of the trunk support exoskeleton of FIG. 1.

FIG. 18 shows an enlarged view of a portion of the trunk support exoskeleton of FIG. 1.

FIG. 19 shows a cross-sectional view of an actuator for the trunk support exoskeleton of FIG. 1.

FIG. 20 shows a cross-sectional view of an actuator for the trunk support exoskeleton of FIG. 1, showing an alternative arrangement of a spring of the actuator.

### DETAILED DESCRIPTION

A trunk support exoskeleton can be used to reduce muscle forces in a wearer's back during lumbar flexion, which occurs during maneuvers such as stooping and bending. A person may wish to wear an exoskeleton to provide support during these activities and also continue wearing the exoskeleton (e.g., for convenience) during other activities, including walking, ascending a slope, or climbing stairs or a ladder. Some embodiments of the present invention provide an exoskeleton that reduces muscle forces in a wearer's back during stooping and bending while also allowing the wearer to comfortably walk, ascend a slope, or climb stairs or a ladder.

In some embodiments, the exoskeleton includes a supporting trunk frame, first and second thigh links, and an actuator that produces an extension torque between the thigh links and supporting trunk frame to provide support to the wearer during stooping or bending.

In some embodiments, the actuator produces an extension torque between the first and second thigh links and the supporting trunk frame only when the wearer is bent forward in a sagittal plane. In some embodiments, the actuator does not produce an extension torque between the first and second thigh links and the supporting trunk frame when a user is walking, ascending, or climbing.

In some embodiments, the actuator generates a torque between a housing of the actuator and a shaft of the actuator, which in turn produces an extension torque between the thigh links and supporting trunk frame. In some embodiments, the torque generated by the actuator between the actuator housing and the actuator shaft is transferred through a system of pulleys and lines to generate the extension torque between the thigh links and supporting trunk frame.

In some embodiments, the actuator housing and actuator shaft can rotate relative to each other (e.g., in response to non-reciprocal motion of thigh links coupled to the actuator housing and actuator shaft, which occurs, e.g., when a wearer's legs do not move in opposite directions at the same speed). Non-reciprocal motion occurs naturally during activities such as walking, ascending, and climbing. For example, during some phases of a walking gait cycle, both legs of a person move in the same direction.

In some embodiments, motion of the first and second thigh links relative to the supporting trunk frame (e.g., while a wearer walks while wearing the trunk support exoskeleton) can transmit forces through a system of lines and pulleys to the actuator housing and actuator shaft. In embodiments in which the actuator housing and actuator shaft can rotate relative to each other, these forces can cause the actuator housing and actuator shaft to rotate relative to each other when the forces are not equal and opposite (i.e., in response to non-reciprocal motion of the thigh links which occurs, for example when a wearer's legs do not move in opposite directions at the same speed). Allowing the actuator housing and actuator shaft to rotate relative to each other in response to non-reciprocal motion of the thigh links can allow a wearer to feel more comfortable when walking, ascending, or climbing while wearing the trunk support exoskeleton. If the actuator housing and actuator shaft could not be rotated relative to each other in response to non-reciprocal motion of the thigh links, tensile forces in the lines connecting the actuator housing and actuator shaft to the first and second thigh links could resist the non-reciprocal motion, making walking, ascending, or climbing uncomfortable or impossible for the user.

In some embodiments, the actuator can rotate relative to the supporting trunk frame (e.g., in response to non-reciprocal motion of thigh links coupled to the actuator housing and actuator shaft).

Allowing the actuator to rotate relative to the supporting trunk frame in response to non-reciprocal motion of the thigh links can allow a wearer to feel more comfortable when walking, ascending, or climbing while wearing the trunk support exoskeleton. If the actuator could not be rotated relative to the supporting trunk frame in response to non-reciprocal motion of the thigh links, tensile forces in the lines connecting the actuator housing and actuator shaft to the first and second thigh links could resist the non-reciprocal motion, making walking, ascending, or climbing uncomfortable or impossible for the user.

These and other embodiments are discussed below in more detail with reference to the figures.

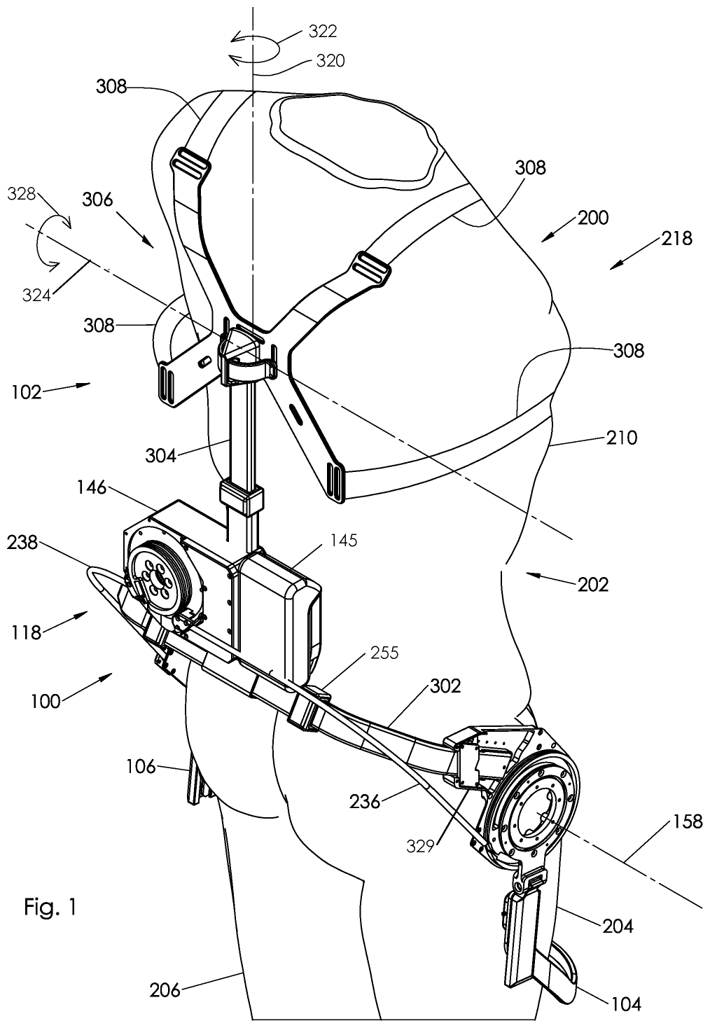

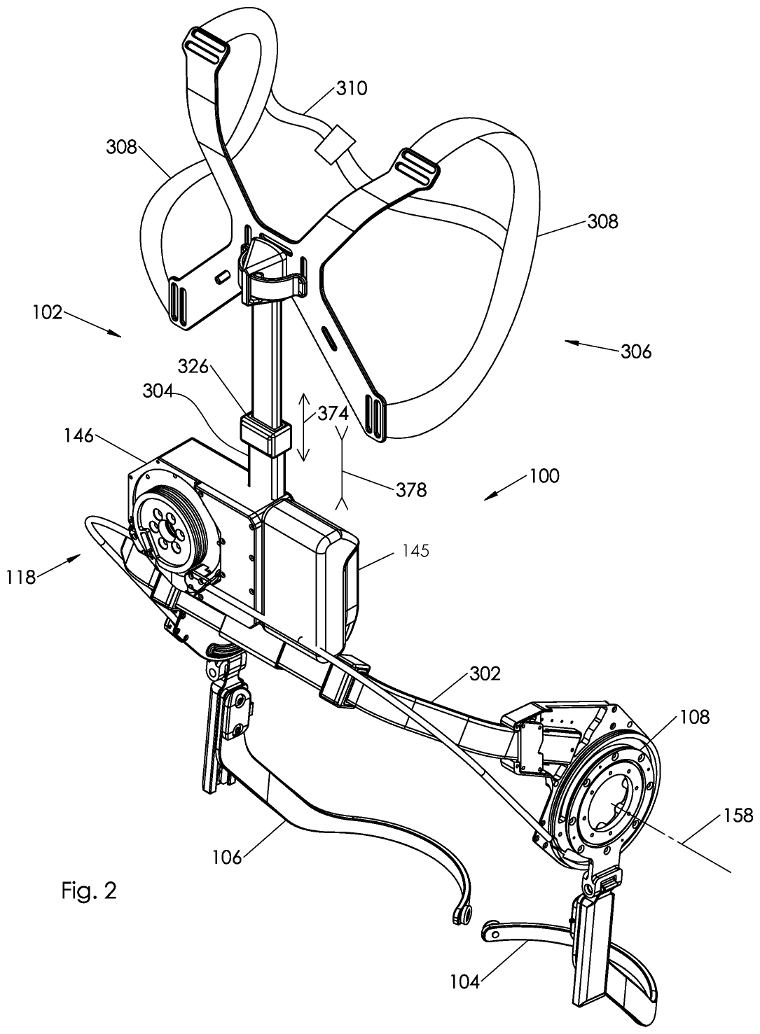

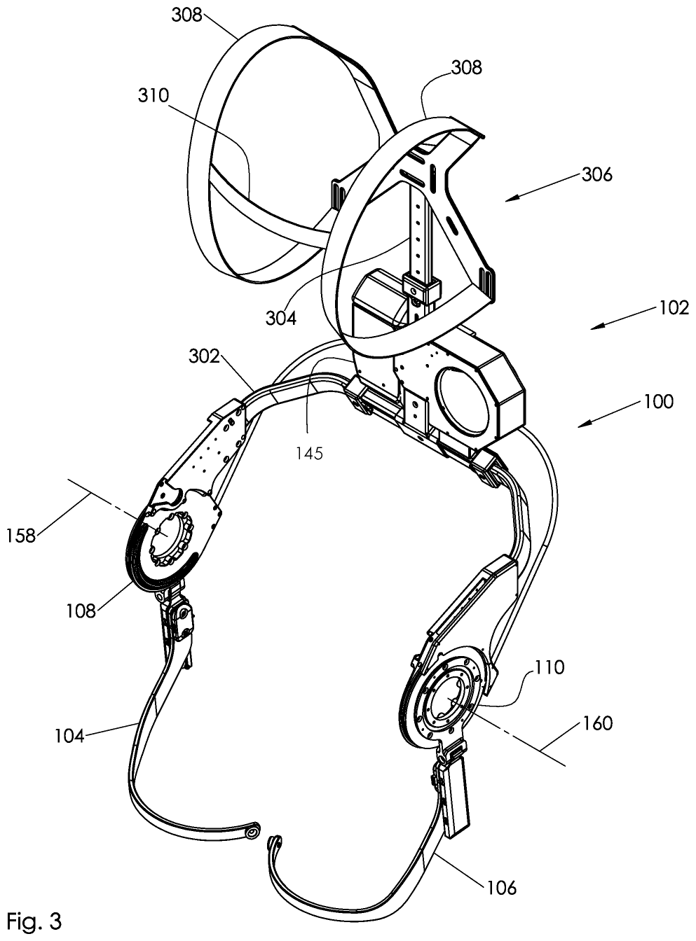

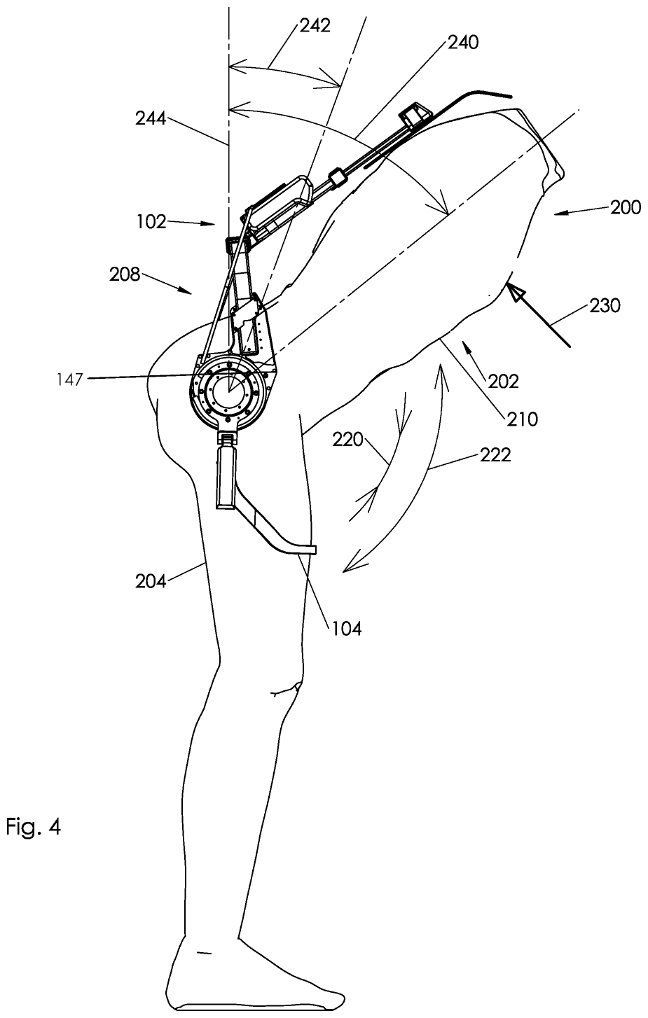

FIGS. 1-4 show an embodiment of a trunk support exoskeleton 100. As will be described, trunk support exoskeleton 100 can be worn by a wearer 200 to reduce muscle forces in the wearer's back during forward lumbar flexion which occurs during maneuvers such as stooping and bending. FIG. 1 shows a perspective view of trunk support exoskeleton 100 worn by a wearer 200. FIG. 2 shows a perspective view of trunk support exoskeleton 100 with wearer 200 removed to further illustrate components of trunk support exoskeleton 100. FIG. 3 shows another perspective view of trunk support exoskeleton 100. FIG. 4 shows wearer 200 wearing trunk support exoskeleton 100 bent forward in a sagittal plane. In this position, forward lumbar flexion is taking place. Angle 240 represents how much wearer 200 has bent along the forward direction.

As shown, for example, in FIG. 1, trunk support exoskeleton 100 can include a supporting trunk frame 102 configured to be coupled to a wearer's trunk 202, a first thigh link 104 and a second thigh link 106 configured to be coupled to respective thighs 204 and 206 of wearer 200, and an actuator 118 that generates an extension torque between first thigh link 104 and supporting trunk frame 102 and between second thigh link 106 and supporting trunk frame 102.

As used here and elsewhere in this disclosure, a wearer's trunk 202 can include the wearer's chest, abdomen, pelvis, and back. The wearer's trunk 202 can be, for example, the wearer's body apart from the head and limbs, or the central part of the wearer from which the neck and limbs extend.

As mentioned, trunk support exoskeleton 100 can include a first thigh link 104 and a second thigh link 106 which are configured to be coupled to respective thighs 204 and 206 of wearer 200. When first thigh link 104 and second thigh link 106 are coupled to respective thighs 204 and 206, first thigh link 104 and second thigh link 106 move in unison with wearer's thighs 204 and 206, respectively, in a manner resulting in flexion and extension of respective first and second thigh links 104 and 106 relative to supporting trunk frame 102.

In some embodiments, first and second thigh links 104 and 106 are rotatably coupled to supporting trunk frame 102 such that the first or second thigh links 104 and 106 can flex or extend relative to supporting trunk frame 102. As shown by arrow 220 in FIG. 4, flexion of first thigh link 104 relative to supporting trunk frame 102 occurs when first thigh link 104 and supporting trunk frame 102 rotate towards each other. Similarly, flexion of second thigh link 106 relative to supporting trunk frame 102 occurs when second thigh link 106 and supporting trunk frame 102 rotate towards each other. As shown by arrow 222 in FIG. 4, extension of first thigh link 104 relative to supporting trunk frame 102 occurs when first thigh link 104 and supporting trunk frame 102 rotate away from each other. Similarly, extension of second thigh link 106 relative to supporting trunk frame 102 occurs when second thigh link 106 and supporting trunk frame 102 rotate away from each other.

As mentioned, and as shown in FIG. 1 and FIG. 2, trunk support exoskeleton 100 can include actuator 118. In some embodiments, as will be discussed, actuator 118 can generate an extension torque between first thigh link 104 and supporting trunk frame 102 and between second thigh link 106 and supporting trunk frame 102 to provide support to the wearer during lumbar flexion. In some embodiments, as will be discussed, actuator 118 is coupled to supporting trunk frame 102 but is free to rotate relative to supporting trunk frame 102.

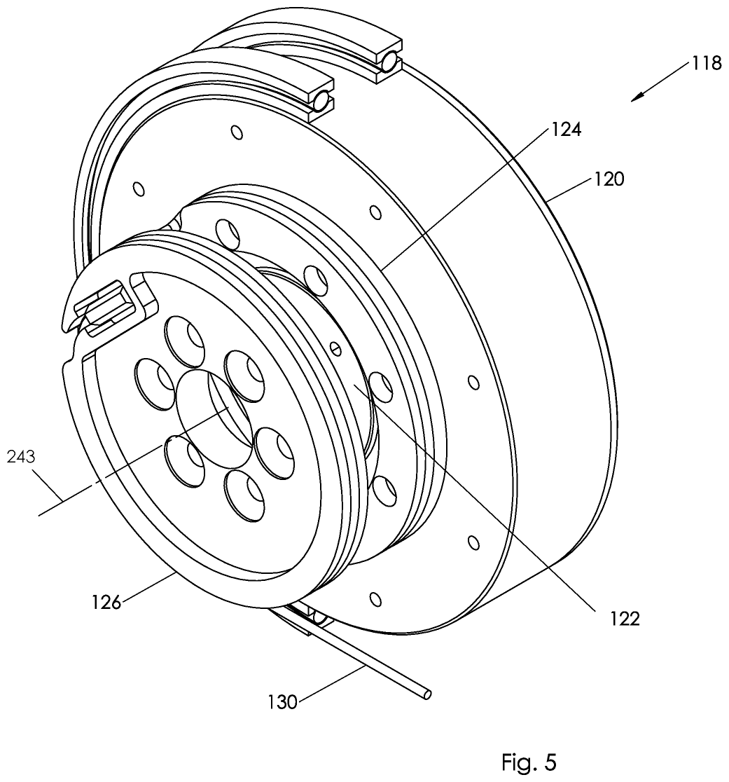

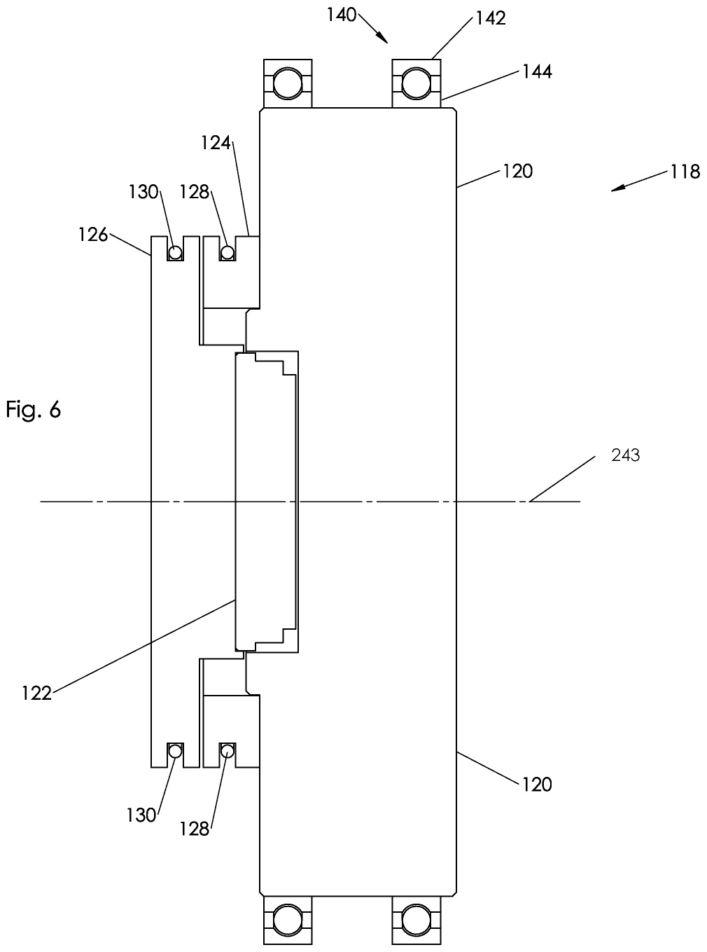

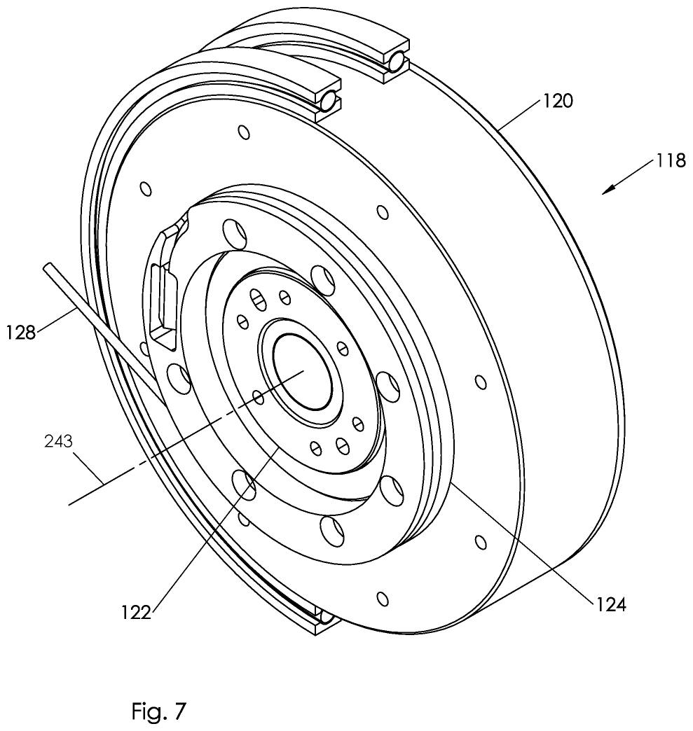

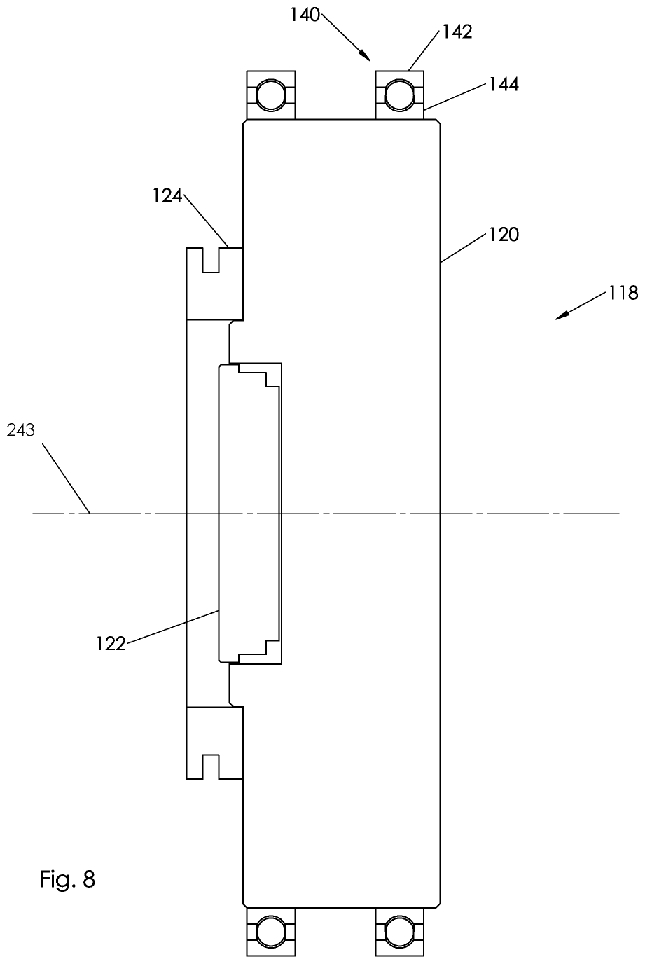

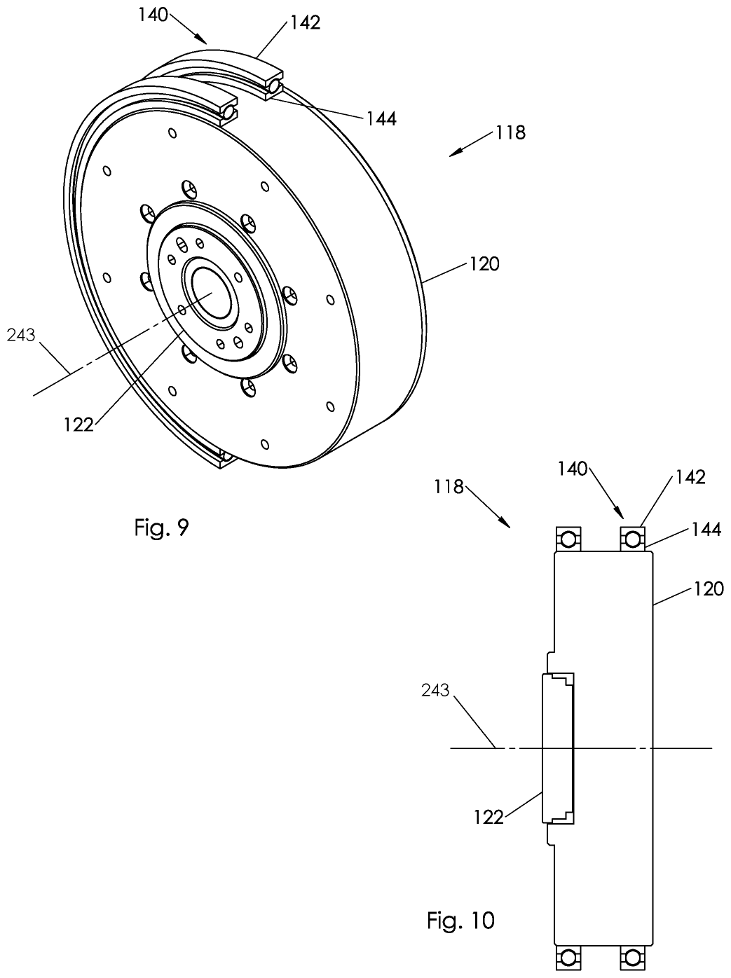

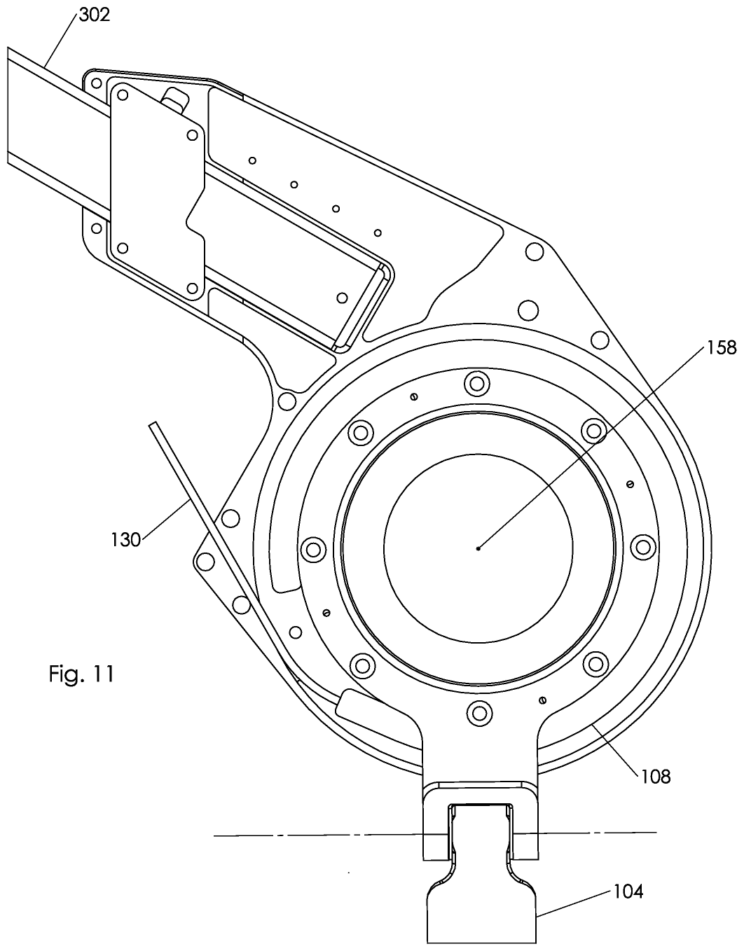

Actuator 118 is discussed below with reference to FIGS. 1 and 5-10. As shown in FIGS. 5 and 6, in some embodiments, actuator 118 includes actuator housing 120, a housing pulley 124, a housing line 128 (shown in FIG. 7), an actuator shaft 122, a shaft pulley 126, and a shaft line 130. In FIGS. 7 and 8, actuator 118 is shown with shaft pulley 126 removed for purposes of illustration. In FIGS. 9 and 10, actuator 118 is shown with housing pulley 124 and shaft pulley 126 removed for purposes of illustration.

In some embodiments, actuator housing 120 and actuator shaft 122 can rotate relative to each other along axis 243. Actuator 118 can be coupled to any point of supporting trunk frame 102. However, both actuator housing 120 and actuator shaft 122 can be free to rotate relative to the supporting trunk frame 102.

In some embodiments, actuator 118 can generate a torque between actuator housing 120 and actuator shaft 122.

As shown in FIGS. 5 and 6, shaft pulley 126 can be coupled to actuator shaft 122 and rotate with actuator shaft 122 (e.g., when actuator 118 generates a torque between actuator housing 120 and actuator shaft 122). Any suitable method can be used to couple shaft pulley 126 and actuator shaft 122 such that shaft pulley 126 and actuator shaft 122 rotate together. For example, fasteners can be used to couple shaft pulley 126 to actuator shaft 122.

As shown in FIGS. 7 and 8, housing pulley 124 can be coupled to actuator housing 120 and rotate with actuator housing 120 along axis 243 (e.g., when actuator 118 generates a torque between actuator housing 120 and actuator shaft 122). Any suitable method can be used to couple housing pulley 124 and actuator housing 120 such that housing pulley 124 and actuator housing 120 rotate together. For example, fasteners can be used to couple housing pulley 124 and actuator housing 120.

In some embodiments, a first end of shaft line 130 can be coupled to (e.g., wound onto) shaft pulley 126, and a second end of shaft line 130 can be coupled to first thigh link 104 such that a tensile force in shaft line 130 (generated, for example, by a torque generated by actuator 118 between actuator housing 120 and actuator shaft 122) generates an extension torque between first thigh link 104 and supporting trunk frame 102.

Shaft line 130 can be or include any device or combination of devices capable of performing the indicated functions. Examples of shaft line 130 include, without limitation, wire, cable, belts, fabric rope, plastic rope, cord, twine, chain, bicycle-type chain, wire rope, string, and combinations thereof. In some embodiments, shaft line 130 comprises a multi-strand wire rope having a maximum strength of about 200 pounds.

In some embodiments, a first end of housing line 128 can be coupled to (e.g., wound onto) housing pulley 124, and a second end of housing line 128 can be coupled to second thigh link 106 such that a tensile force in housing line 128 (generated, for example, by a torque generated by actuator 118 between actuator housing 120 and actuator shaft 122) generates an extension torque between second thigh link 106 and supporting trunk frame 102.

Housing line 128 can be or include any device or combination of devices capable of performing the indicated functions. Examples of housing line 128 include, without limitation, wire, cable, belts, fabric rope, plastic rope, cord, twine, chain, bicycle-type chain, wire rope, string, and combinations thereof. In some embodiments, housing line 128 comprises a multi-strand wire rope having a maximum strength of about 200 pounds.

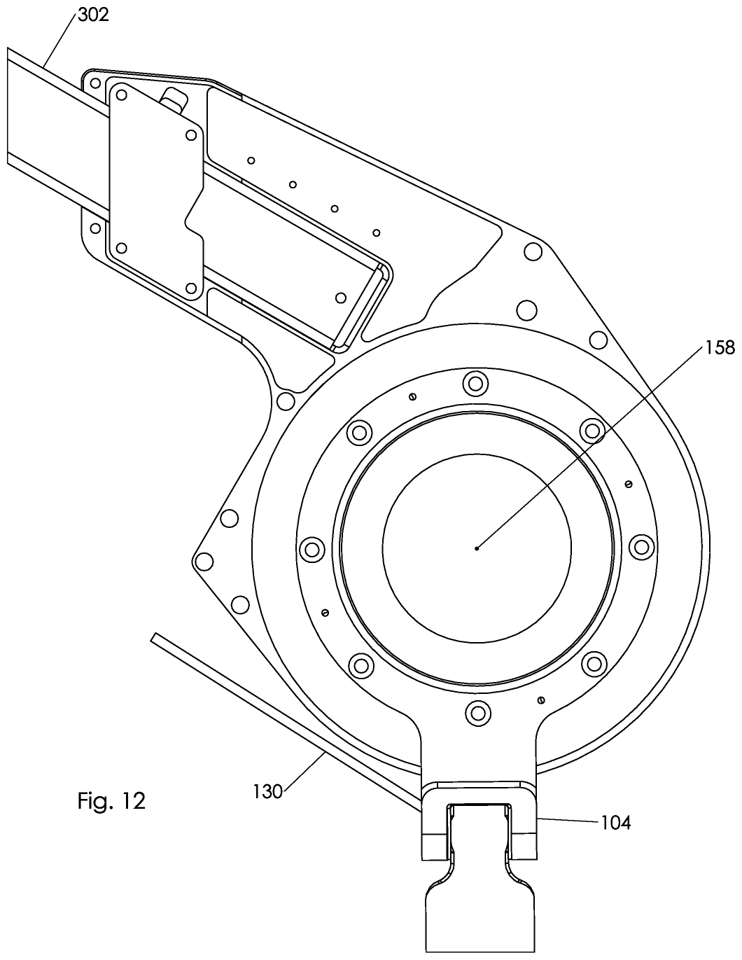

In some embodiments, for example as shown in FIG. 11, first thigh link 104 includes a first thigh link pulley 108 that rotates with first thigh link 104 about axis 158 relative to supporting trunk frame 102. In some embodiments, the second end of shaft line 130 is coupled to (e.g., wound onto) first thigh link pulley 108 such that a tensile force in shaft line 130 generates an extension torque between first thigh link 104 and supporting trunk frame 102. In some embodiments, for example as shown in FIG. 12, the second end of shaft line 130 is directly connected to first thigh link 104 such that a tensile force in shaft line 130 generates a torque about axis 158.

In some embodiments, second thigh link 106 includes a second thigh link pulley 110 that rotates with second thigh link 106 about axis 160 relative to supporting trunk frame 102. In some embodiments, the second end of housing line 128 is coupled to (e.g., wound onto) second thigh link pulley 110 such that a tensile force in housing line 128 generates an extension torque between second thigh link 106 and supporting trunk frame 102. In some embodiments, the second end of housing line 128 is directly connected to second thigh link 106 such that a tensile force in housing line 128 generates a torque about axis 160.

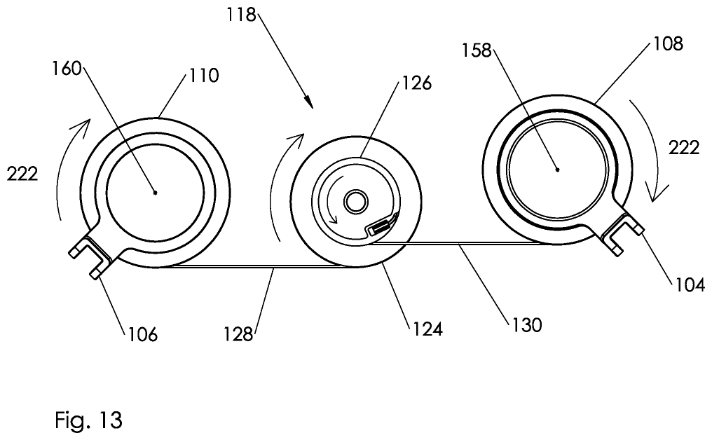

As mentioned, in some embodiments, actuator 118 can generate torque between actuator housing 120 and actuator shaft 122. FIG. 13 schematically shows how this torque can be transferred through the system of pulleys and lines described above. In FIG. 13, the system is flattened into a two-dimensional schematic to show the torque transfer. As shown in FIG. 13, when actuator 118 generates a resisting torque between actuator housing 120 and actuator shaft 122, the torque is transferred to housing pulley 124 (which is coupled to and rotates with actuator housing 120) and shaft pulley 126 (which is coupled to and rotates with actuator shaft 122). In turn, the torque transferred to housing pulley 124 and shaft pulley 126 produce tensile forces in housing line 128 and shaft line 130. These tensile forces provide extension torques between thigh links 104 and 106 and supporting trunk frame 102 in the direction of arrows 222.

In FIGS. 5 and 6, shaft pulley 126 and housing pulley 124 have equal diameters. However, in other embodiments (e.g., as shown in FIG. 13), shaft pulley 126 and housing pulley 124 can have unequal diameters. In the embodiment illustrated in FIGS. 1-4, first thigh link pulley 108 and second thigh link pulley 110 have equal diameters. However, in other embodiments, first thigh link pulley 108 and second thigh link pulley 110 can have unequal diameters. In embodiments in which shaft pulley 126 and housing pulley 124 have equal diameters, and first thigh link pulley 108 and second thigh link pulley 110 have equal diameters, torque between actuator housing 120 and actuator shaft 122 is transferred equally to thigh links 104 and 106. As a result, the extension torque between supporting trunk frame 102 and the thigh links will be equal to twice the torque actuator 118 generates. However, in embodiments in which shaft pulley 126 and housing pulley 124 have unequal diameters, or first thigh link pulley 108 and second thigh link pulley 110 have unequal diameters, unequal torque can transferred to thigh links 104 and 106.

Returning to FIG. 4, in operation, when wearer 200 is bent forward in a sagittal plane such that a predetermined portion 147 of supporting trunk frame 102 passes beyond a predetermined angle 242 from vertical gravitational line 244, actuator 118 can generate a resistive torque between actuator housing 120 and actuator shaft 122. As discussed, this resistive torque can create tensile forces in housing lines 128 and shaft line 130 (e.g., via torque generated between housing pulley 124 and shaft pulley 126), which can then produce extension torques between supporting trunk frame 102 and first and second thigh links 104 and 106. The extension torque between thigh link 104 and supporting trunk frame 102 can try to rotate first thigh link 104 and supporting trunk frame 102 away from each other. Similarly, the extension torque between second thigh link 106 and supporting trunk frame 102 can try to rotate second thigh link 106 and supporting trunk frame 102 away from each other.

The extension torque between supporting trunk frame 102 and first and second thigh links 104 and 106 can impose a supporting trunk force 230 (indicated in FIG. 4) against wearer's trunk 202. Supporting trunk force 230 imposed by supporting trunk frame 102 against wearer's trunk 202 can help reduce the muscle forces at the wearer's lower back at the general area of 208. In the embodiment of FIG. 4, supporting trunk force 230 is generally imposed on wearer's chest area 210. However, supporting trunk force 230 can be imposed on a different portion of wearer's trunk 202 depending on the design of an upper frame part 306 of supporting trunk frame 102. At the same time, first and second thigh links 104 and 106 can impose a force onto wearer's thighs 204 and 206.

In some embodiments, when wearer 200 is not bent forward in the sagittal plane (i.e. when predetermined portion 147 of supporting trunk frame 102 does not pass beyond predetermined angle 242 from vertical gravitational line 244), actuator 118 does not generate a resistive torque between actuator shaft 122 and actuator housing 120. In such embodiments, actuator 118 does not generate an extension torque between supporting trunk frame 102 and first and second thigh links 104 and 106. This means as long as wearer 200 is not bent forward in the sagittal plane, wearer 200 can walk, ascend and descend stairs and ramps without any force imposed on wearer 200 from supporting trunk frame 102.

However, if wearer 200 bends forward in the sagittal plane (i.e. when a predetermined portion 147 of supporting trunk frame 102 passes beyond predetermined angle 242 from vertical gravitational line 244 as shown in FIG. 12), supporting trunk force 230 from supporting trunk frame 102 will help support wearer's trunk 202.

Examples of predetermined angle 242 can be 5, 10 or 15 degrees. In some embodiments, predetermined angle 242 can be zero.

In some embodiments, when wearer 200 is not bent forward in the sagittal plane, actuator 118 generates a substantially small resistive torque between actuator shaft 122 and actuator housing 120. This substantially small resistive torque, generated by actuator 118, can allow for substantially free movement of thigh links 104 and 106 relative to supporting trunk frame 102. This substantially small resistive torque, generated by actuator 118, can cause thigh links 104 and 106 to remain in contact with wearer's thighs during walking. This substantially small resistive torque, generated by actuator 118, can be chosen small enough not to resist or impede the wearer during walking, but cause the thigh links to move in unison with the wearer's thighs.

As mentioned, a person may wish to wear exoskeleton 100 to provide support when bent forward in the sagittal plane as described above and also continue wearing exoskeleton 100 (e.g., for convenience) during other activities, including walking, ascending a slope, or climbing stairs or a ladder. In some embodiments, to enable a wearer to comfortably walk, ascend a slope, or climb stairs or a ladder while wearing exoskeleton 100, actuator housing 120 and actuator shaft 122 can rotate relative to each other along axis 243 in response to non-reciprocal motion of thigh links 104 and 106, which can occur, for example, when a wearer's legs do not move in opposite directions at the same speed.

In some embodiments, actuator housing 120 and actuator shaft 122 can rotate relative to each other in response to non-reciprocal motion of thigh links 104 and 106 when actuator 118 does not generate a resistive torque between actuator housing 120 and actuator shaft 122 (e.g., when wearer 200 is not bent forward in the sagittal plane).

In some embodiments, actuator housing 120 and actuator shaft 122 can rotate relative to each other in response to non-reciprocal motion of thigh links 104 and 106 while actuator 118 generates a substantially small resistive torque between actuator housing 120 and actuator shaft 122.

In some embodiments, extension of first thigh link 104 or second thigh link 106 relative to supporting trunk frame 102 (e.g., when wearer 200 walks) can transmit forces through the system of lines 128, 130 and pulleys 124, 126 described above to actuator shaft 122 and actuator housing 120. In embodiments in which actuator housing 120 and actuator shaft 122 can rotate relative to each other, these forces can cause actuator housing 120 and actuator shaft 122 to rotate relative to each other when the forces are not equal and opposite (e.g., in response to non-reciprocal motion of the wearer's legs). If actuator housing 120 and actuator shaft 122 could not be rotated relative to each other in response to non-reciprocal motion of a person's legs, tensile force in lines 128, 130 connecting actuator housing 120 and actuator shaft 122 to first and second thigh links 104, 106 could resist the non-reciprocal motion of thigh links 104 and 106 relative to supporting trunk frame 102, making walking, ascending, or climbing uncomfortable or impossible for the wearer.

In some embodiments, to enable a wearer to comfortably walk, ascend a slope, or climb stairs or a ladder while wearing exoskeleton 100, actuator 118 is rotatably coupled to supporting trunk frame 102 such that actuator 118 is free to rotate relative to supporting trunk frame 102. In some embodiments, there could be a friction torque that opposes the rotation of actuator 118 relative to supporting trunk frame 102.

Actuator 118 can be coupled to supporting trunk frame 102 via any suitable mechanism that allows actuator 118 to rotate with respect to supporting trunk frame 102. For example, in some embodiments, actuator 118 is coupled to supporting trunk frame 102 via a ball bearing mechanism. As another example, in some embodiments, a bushing can allow rotation of actuator 118 relative to supporting trunk frame 102. In the embodiment illustrated in FIGS. 1-10, for example, an outer race 142 of ball bearing 140 (shown in FIG. 9) is coupled to supporting trunk frame 102, and an inner race 144 of ball bearing 140 is coupled to actuator housing 120. This arrangement allows actuator housing 120 to rotate relative to supporting trunk frame 102.

The coupling of actuator housing 120 to supporting trunk frame 102 is not limited to the arrangement shown. Rather, actuator housing 120 can be coupled to supporting trunk frame 102 via any suitable mechanism that allows actuator housing 120 to rotate with respect to supporting trunk frame 102. One can, for example, use a bushing to allow for rotation of actuator housing 120 relative to supporting trunk frame 102.

As mentioned, in some embodiments, actuator 118 is rotatable relative to supporting trunk frame 102, and in some embodiments, actuator housing 120 and actuator shaft 122 are rotatable relative to each other. In some embodiments, actuator 118 is rotatable relative to supporting trunk frame 102, and actuator housing 120 and actuator shaft 122 are rotatable relative to each other. That is, in some embodiments, actuator housing 120 and actuator shaft 122 are rotatable relative to supporting trunk frame 102 and relative to each other.

In some embodiments, trunk support exoskeleton 100 includes a controller 146 which sends a signal to actuator 118 to generate a resistive torque between actuator housing 120 and actuator shaft 122 (e.g., when wearer 200 is bent forward in the sagittal plane as discussed). Controller 146 can be or include any device or combination of devices capable of performing the indicated functions. Examples of controller 146 include without limitation, analog devices; analog computation modules; digital devices including, without limitation, small-, medium-, and large-scale integrated circuits, application specific integrated circuits, programmable gate arrays, and programmable logic arrays; and digital computation modules including, without limitation, microcomputers, microprocessors, microcontrollers, and programmable logic controllers. In some embodiments controller 146 includes an element or combination of elements selected from a group consisting of electromechanical relays or MOSFET switches.

In some embodiments, trunk support exoskeleton 100 includes a tilt sensor 150 which generates a tilt signal 156. In some embodiments, tilt signal 156 can be indicative of an angle of supporting trunk frame 102 from vertical gravitational line 244 in a sagittal plane. This angle is shown by 240 in FIG. 4.

Tilt sensor 150 can be or include any device or combination of devices capable of performing the indicated functions. Examples of tilt sensor 150 include, without limitation, Inertial Measurement Units (IMU), inclinometers, encoders, and angle sensors.

In operation, controller 146 can send a signal to actuator 118 to generate a resistive torque between actuator housing 120 and actuator shaft 122 when tilt signal 156 indicates that wearer 200 or supporting trunk frame 102 is bent forward in the sagittal plane (i.e., when the tilt signal indicates that an angle of supporting trunk frame 102 from vertical gravitational line 244 in a sagittal plane is greater than predetermined angle 242).

As discussed, the resistive torque generated between actuator housing 120 and actuator shaft 122 can generate tensile forces in housing lines 128 and shaft line 130, and the tensile forces in housing line 128 and shaft line 130 can provide extension torques between supporting trunk frame 102 and thigh links 104 and 106.

The following shows an example calculation of the resistive torque of actuator 118.

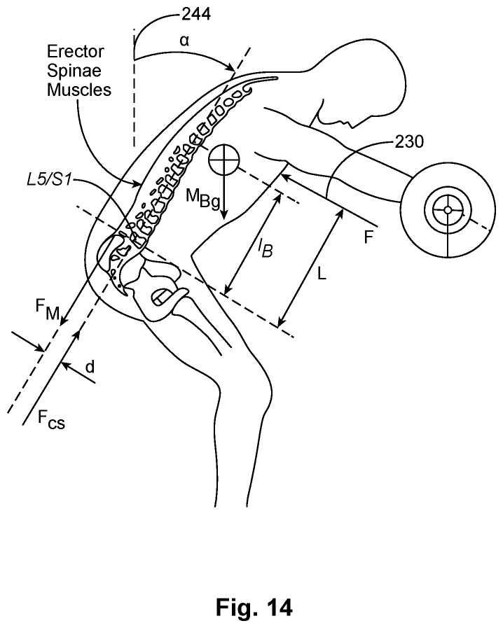

For context, FIG. 14 shows a diagram of forces on a person's back when bending forward in the sagittal plane in the absence of a trunk support exoskeleton. In a static or a quasi-static case and in the absence of any load being lifted by the person, the bending moment (torque) imposed at L5/S1 can be represented by [M_{B} g l_{B} sin(∝)] where M_{B} represents the mass of the person's upper body (including the person's trunk, head and arms), and a part being lifted by person's arms, g represents the gravity acceleration, and l_{B} is the distance between the upper body center of mass and L5/S1 point. ∝ represents the angle of the person's trunk from vertical gravitational line 244. The bending moment increases during load handling and dynamic maneuvers.

As mentioned, when wearer 200 wearing trunk support exoskeleton 100 is bent forward in the sagittal plane, actuator 118 can create an extension torque between supporting trunk frame 102 and first and second thigh links 104 and 106. The extension torque produced by actuator 118 can produce supporting trunk force 230 onto the wearer opposing the bending moment due to the torso and part weight. This means the bending moment (torque) imposed at L5/S1 can be reduced to a new value: [(M_{B}l_{B}) g sin(∝)−FL] where L is the distance from supporting trunk force 230 to point L5/S1 as shown in FIG. 14. This shows the basic concept of a trunk support exoskeleton, where trunk support exoskeleton decreases the bending moment at L5/S1 and consequently decreases the likelihood of injuries during repetitive maneuvers.

Erector spinae muscle tensile force F_{M} decreases as supporting trunk force 230 increases. In a more general case, erector spinae muscle force F_{M}, with the angular speed and acceleration of \dot{∝} and \ddot{∝}, can be expressed as:

$${F}_{M} = ( {M}_{B} {l}_{B} ) \frac{g}{d} sin ( α ) - \frac{FL}{d} - \ddot{∝} \frac{I}{d} - C \dot{∝} \frac{1}{d} \qquad ( 1 )$$

C is a constant and C \dot{∝} represents the velocity dependent torque. I is the effective moment of inertia of the upper body and \ddot{∝}I represents the acceleration dependent torque.

Spine compression force F_{CS} similarly decreases as supporting trunk force 230 is increased and can be expressed as:

$${F}_{CS} = ( {M}_{B} {l}_{B} ) \frac{g}{d} sin ( ∝ ) + ( {M}_{B} ) g cos ( ∝ ) - ( {M}_{B} {l}_{B} ) {\dot{∝}}^{2} - \ddot{∝} \frac{I}{d} - \dot{∝} \frac{C}{d} - \frac{FL}{d} \qquad ( 2 )$$

This analysis assumes F_{CS} and F_{M} to act perpendicularly to supporting trunk force 230. In theory, if the exoskeleton supporting torque FL is chosen as equation (3), then force F_{M}, reduces to zero (equation 4) and force F_{CS}, reduces substantially (equation 5):

$$F L = ( {M}_{B} {l}_{B} ) g sin ( ∝ ) - I \ddot{∝} - C \dot{∝} \qquad ( 3 )$$

$${F}_{M} = 0 \qquad ( 4 )$$

$${F}_{CS} = ( {M}_{B} ) g cos ( ∝ ) - ( {M}_{B} {l}_{B} ) {( \dot{∝} )}^{2} \qquad ( 5 )$$

This means, in theory, it is possible to reduce the erector spinae muscle force, F_{M}, to zero and the spine compression force, F_{CS}, to a smaller value by controlling supporting trunk force 230 acting on wearer 200. However, the parameters in equation (3) can be difficult to measure or calculate precisely. For example, M_{B} is not a known quantity, l_{B} is simply estimates, and the measurements of ∝ is not precise. Nevertheless, any attempt to cancel the terms of equation (1) by use of equation (3) will lead to a reduction of the spine compression force, F_{CS} and erector spinae muscle force F_{M}. In most cases, bending acceleration \ddot{∝} is negligible. As these equations demonstrate, when supporting trunk force 230 is increased, both erector spinae muscle force and the spine compression force (shown by F_{CS} and F_{M}) are decreased.

The term F L is referred to as supporting torque because it supports wearer 200 during bending and stooping. As can be seen from FIG. 14, this supporting torque is an extension torque. In some embodiments, exoskeleton supporting torque F L can be chosen as

$$F L = {K}_{1} sin ( ∝ ) = {K}_{2} \dot{∝} \qquad ( 6 )$$

K₁ and, K₂ represent approximate values of parameters of equation (3) if acceleration \ddot{∝} is negligible. As shown by equation (6), the exoskeleton supporting torque FL, in some embodiments, comprises a torque which is a function of angle of ∝. In some embodiments, as shown by equation (6), the exoskeleton supporting torque FL comprises a torque which is a function of the angular speed of the supporting trunk frame \dot{∝}. The acceleration dependent term, I \ddot{∝}, in some applications is small and can be neglected. If \ddot{∝} can be measured or estimated with little noise, then the inclusion of I \ddot{∝}, in equation (6) can improve the device performance.

If shaft pulley 126 and housing pulley 124 have equal diameters, and if first thigh link pulley 108 and second thigh link pulley 110 have equal diameters, then the resistive torque of actuator 118, indicated by T_{R}, is represented by equation (7)

$${T}_{R} = \frac{[ F L ]}{2} = [ {K}_{1} sin ( ∝ ) - {K}_{2} \dot{∝} ] / 2 \qquad ( 7 )$$

In some embodiments, the resistive torque, as shown by equation 7, is a function of tilt signal 156. In some embodiments, the resistive torque is a function of how much the wearer is bent forward in the sagittal plane. In some embodiments, the resistive torque increases as the angle of the supporting trunk frame 102 from vertical gravitational line 244 increases. In some embodiments, the resistive torque decreases as the angle of the supporting trunk frame 102 from vertical gravitational line 244 decreases. In some embodiments, the resistive torque is a function of the angular velocity of supporting trunk frame 102 in the sagittal plane. In some embodiments, the resistive torque decreases as the forward angular velocity of supporting trunk frame 102 in the sagittal plane increases. This can, for example, allow wearer 200 to bend forward in the sagittal plane with little effort to push against supporting trunk frame 102. In some embodiments, the resistive torque increases as the forward angular velocity of supporting trunk frame 102 in the sagittal plane decreases. In some embodiments, the resistive torque decreases as the backward angular velocity of the supporting trunk frame 102 in the sagittal plane increases. In some embodiments, the resistive torque increases as the backward angular velocity of supporting trunk frame 102 in the sagittal plane decreases.

In some embodiments, controller 146 stops sending a signal to actuator 118 to generate resistive torque according to equation (7) when tilt signal 156 indicates that wearer 200 is no longer bent forward in the sagittal plane.

In some embodiments, controller 146 sends a signal to actuator 118 to generate a substantially small resistive torque between actuator housing 120 and actuator shaft 122 when tilt signal 156 indicates that wearer 200 is not bent forward in the sagittal plane.

In some embodiments, controller 146 sends a signal to actuator 118 to generate a zero torque when tilt signal 156 indicates that wearer 200 is not bent forward in the sagittal plane.

In some embodiments, actuator 118 provides a small amount of tensile force in housing line 128 and shaft line 130 by providing a biased resistive torque, T_{biased}, in equation 7., T_{biased}, shown in equation 8, causes leads to small initial tensile force in housing line 128 and shaft line 130.

$${T}_{R} = {T}_{biased} + [ {K}_{1} sin ( ∝ ) - {K}_{2} \dot{∝} ] / 2 \qquad ( 8 )$$

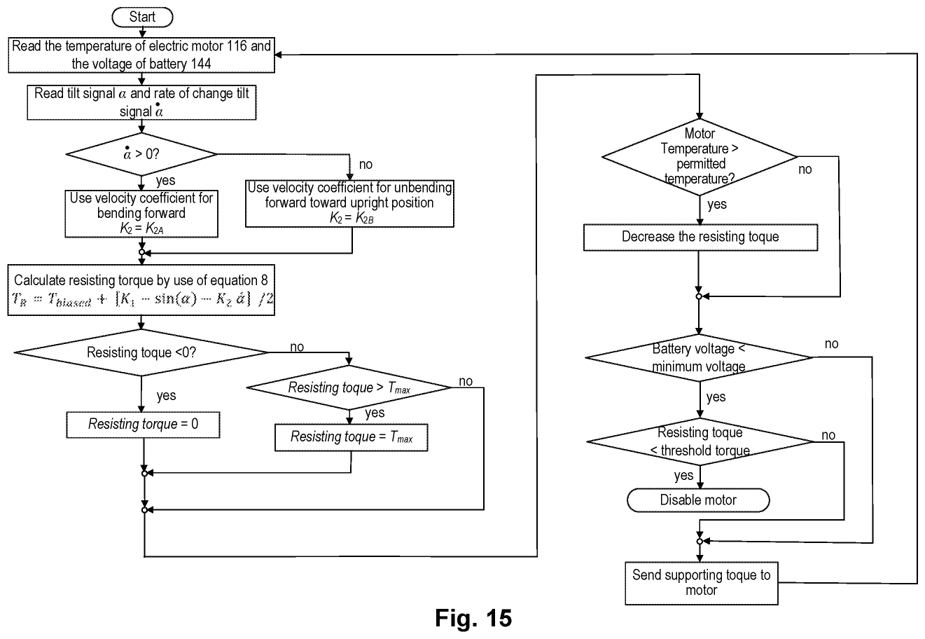

FIG. 15 shows a flowchart of a control algorithm for exoskeleton 100. The control software can start by reading one or more of the voltage of battery 145, the temperature of actuator 118 or a component thereof (e.g., an electric motor 116), tilt signal 156, or the rate of change of tilt signal 156.

In embodiments in which the voltage of battery 145 is checked, if the voltage of battery 145 is less than a minimum voltage, and the calculated resistive toque is smaller than a threshold torque, then actuator 118 can be disabled.

In embodiments in which the actuator temperature is checked, if the actuator temperature is larger than a permitted temperature, the resistive torque can be decreased.

In embodiments in which tilt signal 156 and/or the rate of change of tilt signal 156 is read, a resistive torque for actuator 118 can be calculated using equation 8. If \dot{α} is positive (i.e. wearer 200 is bending forward in the sagittal plane,) coefficient K₂, (used in equation 6) can be chosen as K_{2F}. Otherwise, coefficient K₂ can be chosen as K_{2B}. In some embodiments, K_{2F} is larger than K_{2B}. This allows the resistive torque to be smaller when bending forward in the sagittal plane. The values of K_{2F} and K_{2B} can be chosen to provide appropriate comfort for wearer 200.

In some embodiments, the calculated resistive torque can be checked to see if it is negative or positive. In some embodiments, if the calculated resisting toque is negative, the resistive torque can be set to zero.

In some embodiments, the resistive torque can be checked to see if it is larger than a maximum torque of actuator 118 (or a maximum torque that can be generated by electric motor 116 of actuator 118). This maximum torque is referred to as Tₘₐₓ. If the calculated value of the resisting torque is larger than the maximum torque Tₘₐₓ, then the resistive torque can be set as Tₘₐₓ.

Additional aspects relating to supporting trunk frame 102 will now be discussed with reference to FIG. 1 and FIGS. 16-18. As shown in FIG. 1, in some embodiments, supporting trunk frame 102 includes a lower frame part 302, a spine frame part 304, and an upper frame part 306.

As shown in FIG. 1, in some embodiments, lower frame part 302 is substantially located behind wearer 200 when trunk support exoskeleton 100 is worn. In some embodiments, lower frame part 302 is configured to partially surround wearer's trunk 202 and hips. In some embodiments, lower frame part 302 is coupled to first and second thigh links 104 and 106 from two sides of wearer 200.

Spine frame part 304 can be coupled to (e.g., rotatably coupled to) lower frame part 302. In some embodiments, spine frame part 304 is rotatable about axis 320 with respect to lower frame part 302. This can, for example, allow wearer 200 to freely rotate his upper body relative to his lower body. In some embodiments, axis 320 is substantially parallel to the wearer's spine. Arrow 322 shows the direction of rotation of spine frame part 304 relative to lower frame part 302 about axis 320. In some embodiments, spine frame part 304 is located behind wearer 200 when trunk support exoskeleton 100 is worn.

Upper frame part 306 can be coupled to (e.g., rotatably coupled to) spine frame part 304. In some embodiments, upper frame part 306 is rotatable about axis 320 with respect to spine frame part 304. This can, for example, allow wearer 200 to freely rotate his upper body relative to his lower body. In some embodiments, axis 320 is substantially parallel to the wearer's spine. Arrow 322 shows the direction of rotation of upper frame part 306 relative to spine frame part 304 about axis 320. In some embodiments, upper frame part 306 is rotatable about axis 324 relative to spine frame part 304. This can, for example, allow wearer 200 to freely rotate his upper body relative to his lower body. In some embodiments, axis 324 is substantially parallel to one of the wearer's lumbar spine mediolateral flexion and extension axes. Arrow 328 shows the direction of rotation of upper frame part 306 relative to spine frame part 304 about axis 324.

In some embodiments, upper frame part 306 is configured to contact a wearer's trunk 202 such that upper frame part 306 can impose a force (e.g., supporting trunk force 230 shown in FIG. 6) on a front part of wearer's trunk 202. In some embodiments, upper frame part 306 is configured to contact a chest area 210 of wearer's trunk 202 such that upper frame part 306 can impose a force (e.g., supporting trunk force 230 shown in FIG. 4) on a chest area of wearer's trunk 202. In some embodiments, upper frame part 306 is configured to contact a shoulder area 218 of wearer's trunk 202 such that upper frame part 306 can impose a force (e.g., supporting trunk force 230) on a shoulder area 218 of wearer's trunk 202. As shown in FIG. 2 and FIG. 3, in some embodiments, upper frame part 306 includes shoulder straps 308. In some embodiments, as shown in FIG. 2 and FIG. 3, upper frame part 306 includes chest straps 310.

As mentioned, in some embodiments, spine frame part 304 is rotatable with respect to lower frame part 302, and in some embodiments, upper frame part 306 is rotatable with respect to spine frame part 304. In some embodiments, both upper frame part 306 is rotatable with respect to spine frame part 304 and spine frame part 304 is rotatable with respect to lower frame part 302.

In some embodiments, a height of supporting trunk frame 102 is adjustable. In some embodiments, supporting trunk frame 102 includes an adjustment mechanism 326 (shown in FIG. 2) to adjust the height of supporting trunk frame 102. For example, in some embodiments, upper frame part 306 is configured to slide linearly along spine frame part 304 to adjust a height supporting trunk frame 102. In some embodiments, a height of supporting trunk frame 102 can be increased or decreased as shown by arrows 374 and 378 in FIG. 2.

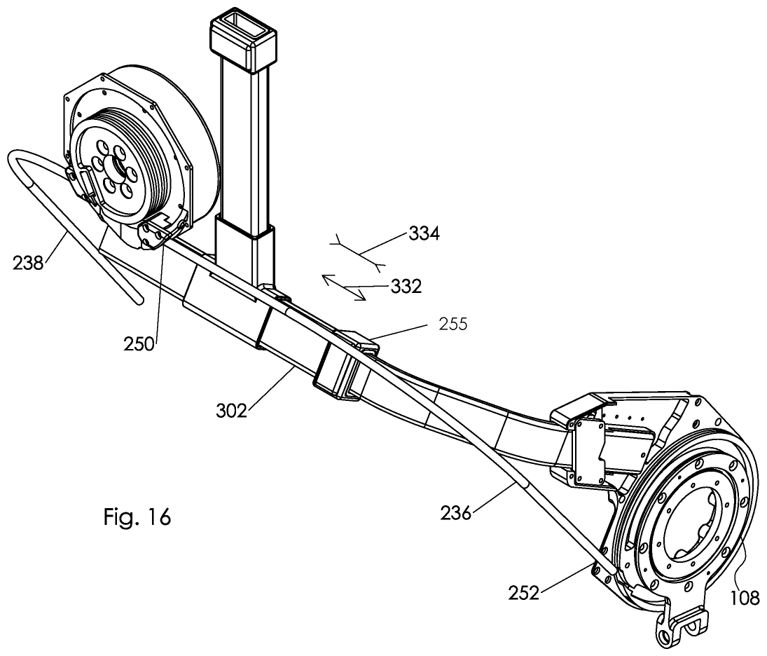

In some embodiments, lower frame part 302 is adjustable in width to fit various people. In some embodiments, supporting trunk frame 102 includes an adjustment mechanism 255 (shown in FIG. 16) to adjust a width of lower frame part 302. For example, in some embodiments, adjustment mechanism 255 can increase or decrease a width of lower frame part 302 as shown by arrows 332 and 334 in FIG. 16.

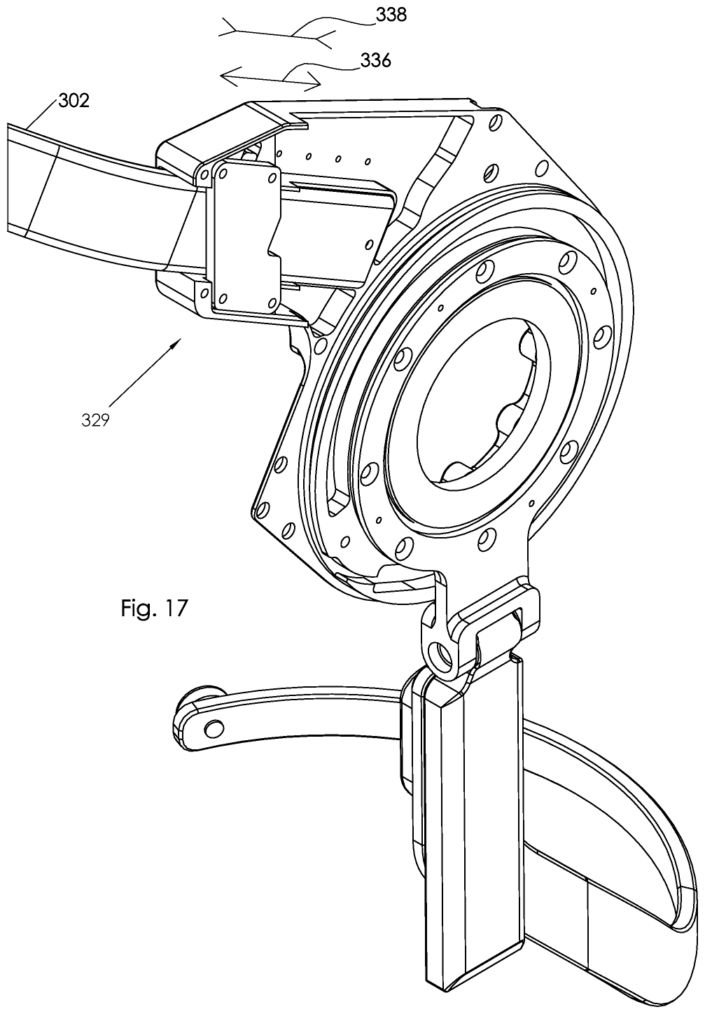

In some embodiments, lower frame part 302 is adjustable in depth to fit various people. In some embodiments, supporting trunk frame 102 includes an adjustment mechanism 329 (shown in FIG. 17) to adjust a depth of lower frame part 302. For example, in some embodiments, adjustment mechanism 329 can increase or decrease a depth of lower frame part 302 as shown by arrows 336 and 338 in FIG. 17.

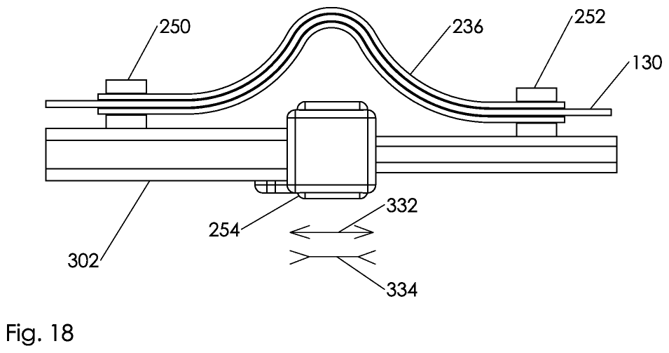

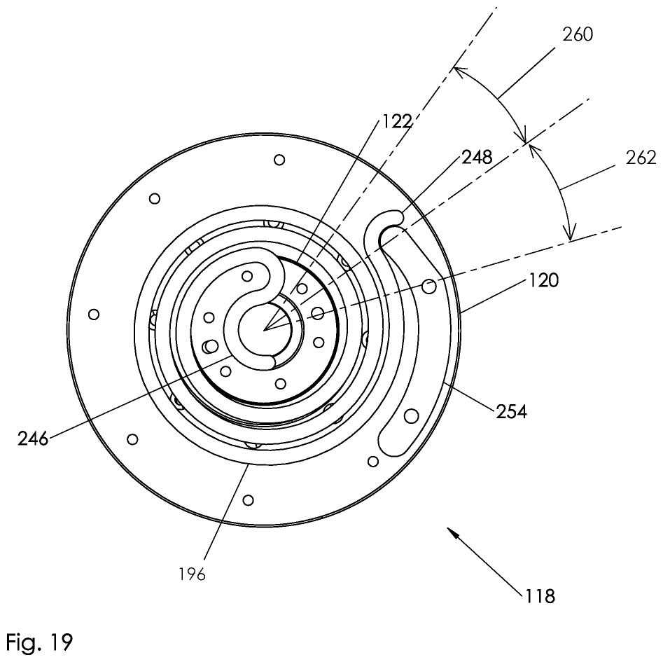

As mentioned, in some embodiments, supporting trunk frame 102 can be adjustable in width, length and depth to fit various wearers. In some such embodiments, and as shown in FIGS. 16 and 18, a shaft line jacket 236 encloses shaft line 130 and is secured to supporting trunk frame 102 at locations 250 and 252. This arrangement can help facilitate size adjustment of supporting trunk frame 102 without needing to adjust the size of shaft line 130. Similarly, in some embodiments, a housing line jacket 238 encloses housing line 128 and is secured to supporting trunk frame 102. This arrangement can help facilitate size adjustment of supporting trunk frame 102 without need to adjust the size of housing line 128. FIG. 18 schematically shows how shaft line 130 can be framed by jacket 236.

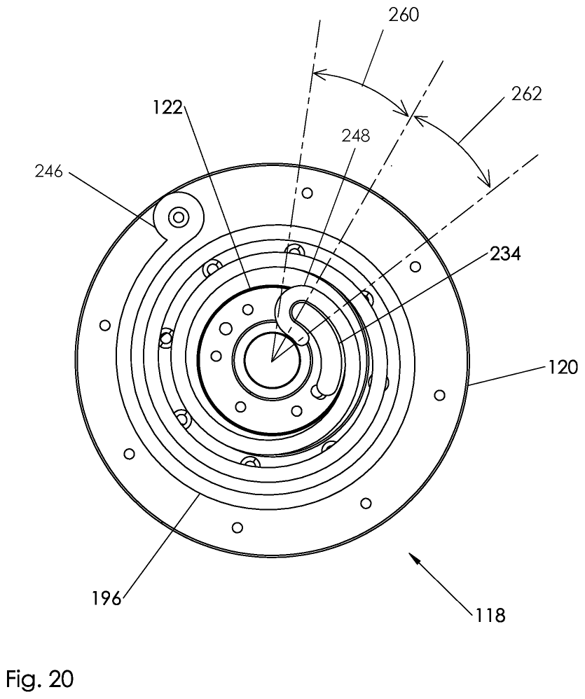

Additional aspects relating to actuator 118 will now be discussed with reference to FIGS. 19 and 20.

Actuator 118 can be or include any device or combination of devices capable of performing the indicated functions. Examples of actuator 118 include, electric motors, including, without limitation, AC (alternating current) motors, brush-type DC (direct current) motors, brushless DC motors, electronically commutated motors (ECMs), stepping motors, and combinations thereof. In some embodiments, actuator 118 comprises transmission systems such as harmonic drives, planetary gears, ball screw mechanism, lead screw mechanism, worm gear and combinations thereof. In some embodiments, actuator 118 comprises hydraulic actuators.

In some embodiments, actuator 118 includes an electric motor 116 configured to generate torque between actuator shaft 122 and actuator housing 120 by use of electric power. Electric motor 116 can be or include any device or combination of devices capable of performing the indicated functions. Examples of electric motor include, without limitation, an element or combination of elements selected from a group consisting of electric motors including, without limitation, AC (alternating current) motors, brush-type DC (direct current) motors, brushless DC motors, electronically commutated motors (ECMs), stepper motors, and combinations.

In some embodiments, actuator 118 includes a transmission system to alter and condition the torque of actuator 118. The transmission system can be or include any device or combination of devices capable of performing the indicated functions. Examples of transmission system include, without limitation, gear, worm gears, gear trains, pulleys, lines, belts, toothed belts, toothed pulleys, planetary gears, harmonic drives, spur gears, flexible belt, wire ropes, ropes, ball screw mechanisms, and lead screw mechanisms.

In some embodiments, as shown in FIGS. 19 and 20, actuator 118 includes an actuator spring 196 to generate resistive torque between actuator shaft 122 and actuator housing 120.

In some embodiments, actuator 118 includes both an electric motor 116 and an actuator spring 196 to generate resistive torque between actuator shaft 122 and actuator housing 120.

As shown in FIG. 19, actuator spring 196 can include a first end 246 and a second end 248. First end 246 of actuator spring 196 can be coupled, either directly or indirectly, to actuator shaft 122 such that when actuator shaft 122 turns relative to actuator housing (e.g., in response to wearer 200 bending in the sagittal plane), first end 246 of actuator spring 196 turns with actuator shaft 122.

When actuator shaft 122 is in a first range of rotation 260 of actuator shaft 122 relative to actuator housing 120, second end 248 of actuator spring 196 can be free. When actuator shaft 122 rotates relative to actuator housing 120 beyond the first range of rotation (e.g., by rotating in a clockwise direction relative to actuator housing 120), second end 248 of actuator spring 196 can become constrained by actuator housing 120. In other words, in this second range of rotation, second end 248 of actuator spring 196 can be coupled directly or indirectly to actuator housing 120 so that second end 248 of actuator spring 196 turns with actuator housing 120. When actuator shaft 122 is in this second range of rotation 262 in which second end 248 of spring 196 is constrained, further rotation of actuator shaft 122 relative to actuator housing 120 in the same direction (e.g., clockwise) can cause spring 196 to deflect and thereby cause spring 196 to provide a spring resistive torque on actuator shaft 122. In some embodiments, actuator housing 120 can include a housing protrusion 254 (shown in FIG. 19) to constrain second end 248 of actuator spring 196. As shown in FIG. 19, when actuator shaft 122 rotates relative to actuator housing 120 (e.g., in a clockwise direction), second end 248 becomes engaged with housing protrusion 254. After engagement of second end 248 with housing protrusion 254, second end 248 no longer rotates relative to actuator housing 120. Further rotation of actuator shaft 122 relative to actuator housing 120 in the same direction causes actuator spring 196 to flex and resist the rotation of actuator shaft 122 relative to actuator housing 120.

The configuration described above can allow for generation of the resistive torque not only by use of electric power, but also passively by use of actuator spring 196. In operation, when actuator shaft 122 is in the first range of rotation, electric motor 116 can generate resistive torque between actuator shaft 122 and actuator housing 120 by use electric power. When actuator shaft 122 is in the second range of rotation, actuator spring 196 can provide a spring resistive torque between actuator shaft 122 and actuator housing 120. In some embodiments, when actuator shaft 122 is in the second range of rotation, electric motor 116 can also provide a motor resistive torque on actuator shaft 122 relative to actuator housing 120. In such embodiments, the resistive torque is the addition of both spring torque and the torque generated by motor 116. The technique of adding an actuator spring torque in parallel with the electric motor torque discussed above is useful in increasing the torque capability of actuator 118.

Examples of actuator spring 196 include, without limitation, coil spring, rotary spring, leaf spring, helical spring, bungee cord, elastomer cord, elastic cord, elastic fabric cord, plastic cord, elastomer cord, twine, wire rope elastomer, string, and combinations thereof.

In the embodiment shown in FIG. 19, first end 246 of spring 196 is coupled to actuator shaft 122. However, in other embodiments, for example in the embodiment shown in FIG. 20, first end 246 end of spring 196 is instead coupled to actuator housing 120 such that when actuator housing 120 turns relative to actuator shaft 122 (e.g., in response to wearer 200 bending in the sagittal plane), first end 246 of actuator spring 196 turns with actuator housing 120. As shown in FIG. 20, when actuator housing 120 is in a first range of rotation 260 relative to actuator shaft 122, second end 248 of spring 196 can be free to rotate. When actuator housing 120 rotates relative to actuator shaft 122 (e.g., in a clockwise direction) beyond first range of rotation 260, second end 248 of spring 196 can become constrained (e.g., by a shaft constraining element 234 which is coupled to actuator shaft 122).

Based on the two embodiments of FIG. 19 and FIG. 20, it can be observed that, in general, spring 196 includes a first end 246 and a second end 248. First end 246 of spring 196 can be coupled to one of actuator shaft 122 or actuator housing 120. When actuator shaft 122 is in a first range of rotation relative to actuator housing 120, second end 248 of spring 196 can be free. Thus, when actuator shaft 122 is in the first range of rotation, spring 196 does not provide a spring resistive torque on actuator shaft 122. When actuator shaft 122 is in the second range of rotation relative to actuator housing 120, second end 248 of spring 196 can be constrained by the other one of actuator shaft 122 or actuator housing 120. In operation, when actuator shaft 122 is in the first range of rotation, motor 116 can provide a motor resistive torque on actuator shaft 122 relative to actuator housing 120. When actuator shaft 122 is in the second range of rotation, spring 196 can provide a spring resistive torque on actuator shaft 122 relative to actuator housing 120. In some embodiments, when actuator shaft 122 is in the second range of rotation, motor 116 can also provide a motor resistive torque on actuator shaft 122 relative to actuator housing 120.

1.-41. (canceled)

42. A trunk supporting exoskeleton for reducing muscle forces in a wearer's back during forward lumbar flexion, the trunk supporting exoskeleton comprising:
a supporting trunk frame configured to be coupled to the wearer's trunk;
a first thigh link configured to be coupled to one of the wearer's thighs;
a second thigh link configured to be coupled to another of the wearer's thighs, wherein each of the first and second thigh links is rotatably coupled to the supporting trunk frame such that the respective first or second thigh links can flex or extend relative to the supporting trunk frame;
an actuator coupled to the supporting trunk frame, wherein the actuator comprises an actuator housing and an actuator shaft, wherein the actuator shaft and the actuator housing are rotatable relative to the supporting trunk frame;
a shaft pulley coupled to the actuator shaft;
a housing pulley coupled to the actuator housing;
a shaft line having a first end wound onto the shaft pulley and a second end coupled to the first thigh link; and
a housing line having a first end wound onto the housing pulley and a second end coupled to the second thigh link,
wherein when the wearer is bent forward relative to a vertical gravitational line in a sagittal plane, the actuator generates an actuator resistive torque between the actuator housing and the actuator shaft, and
wherein the actuator resistive torque between the actuator housing and the actuator shaft generates tensile forces in the housing line and the shaft line, thereby generating extension torques between the respective first and second thigh links and the supporting trunk frame.

43. The trunk supporting exoskeleton of claim 42, wherein:
the first thigh link comprises a first thigh link pulley,
the second thigh link comprises a second thigh link pulley,
the second end of the shaft line is wound onto the first thigh link pulley such that a tensile force in the shaft line provides an extension torque between the first thigh link and the supporting trunk frame, and
the second end of the housing line is wound onto the second thigh link pulley such that a tensile force in the housing line provides an extension torque between the second thigh link and the supporting trunk frame.

44. The trunk supporting exoskeleton of claim 42, wherein when the wearer is not bent forward relative to the vertical gravitational line, the actuator does not produce an actuator resistive torque between the actuator housing and the actuator shaft.

45. The trunk supporting exoskeleton of claim 42, wherein when the wearer is not bent forward relative to the vertical gravitational line, the actuator generates a substantially small actuator resistive torque between the actuator housing and the actuator shaft allowing for substantially free movement of the thigh links.

46. The trunk supporting exoskeleton of claim 42, wherein when the wearer is not bent forward relative to the vertical gravitational line and the thigh links are in a reciprocating mode indicative of walking, the actuator generates a substantially small actuator resistive torque allowing for substantially free movement of the thigh links.

47. The trunk supporting exoskeleton of claim 42, further comprising a controller configured to send a signal to the actuator to generate the actuator resistive torque between the actuator housing and the actuator shaft when the wearer is bent forward relative to the vertical gravitational line.

48. The trunk supporting exoskeleton of claim 47, wherein the controller sends a signal to the actuator to generate a substantially small actuator resistive torque between the actuator housing and the actuator shaft when the wearer is not bent forward relative to the vertical gravitational line.

49. The trunk supporting exoskeleton of claim 42, wherein the actuator resistive torque increases as an angle of the supporting trunk frame relative to the vertical gravitational line increases.

50. The trunk supporting exoskeleton of claim 42, wherein the actuator resistive torque decreases as an angle of the supporting trunk frame relative to the vertical gravitational line decreases.

51. The trunk supporting exoskeleton of claim 42, wherein the actuator resistive torque is a function of an angular velocity of the supporting trunk frame in the sagittal plane.

52. The trunk supporting exoskeleton of claim 42, wherein the actuator resistive torque decreases as a forward angular velocity of the supporting trunk frame in the sagittal plane increases.

53. The trunk supporting exoskeleton of claim 42, wherein the actuator resistive torque increases as a forward angular velocity of the supporting trunk frame in the sagittal plane decreases.

54. The trunk supporting exoskeleton of claim 42, wherein the actuator resistive torque decreases as a backward angular velocity of the supporting trunk frame in the sagittal plane decreases.

55. The trunk supporting exoskeleton of claim 42, wherein the actuator resistive torque increases as a backward angular velocity of the supporting trunk frame in the sagittal plane decreases.

56. The trunk supporting exoskeleton of claim 42, further comprising:
a tilt sensor that generates a tilt signal indicative of an angle of the supporting trunk frame relative to the vertical gravitational line in the sagittal plane; and
a controller configured to send a signal to the actuator to generate the actuator resistive torque between the actuator housing and the actuator shaft when the tilt signal indicates that the angle of the supporting trunk frame relative to the vertical gravitational line is greater than a predetermined angle.

57. The trunk supporting exoskeleton of claim 42, wherein:
the actuator comprises an actuator spring,
a first end of the actuator spring is coupled to the actuator shaft,
a second end of the actuator spring is free in a first range of rotation of the actuator shaft relative to the actuator housing,
a second end of the actuator spring is constrained by the actuator housing in a second range of rotation of the actuator shaft relative to the actuator housing,
in the first range of rotation, the actuator generates the actuator resistive torque by use of electric power, and
in the second range of rotation, the spring generates at least part of the actuator resistive torque.

58. The trunk supporting exoskeleton of claim 42, wherein in a first range of rotation of the actuator shaft relative to the actuator housing the actuator generates the actuator resistive torque by use of electric power, and wherein in a second range of rotation of the actuator shaft relative to the actuator housing, the actuator resistive torque comprises a torque generated by a spring and a torque generated by use of the electric power.

59. The trunk supporting exoskeleton of claim 42, further comprising:
a shaft line jacket enclosing the shaft line, wherein the shaft line jacket is secured to the supporting trunk frame to facilitate a size adjustment of the supporting trunk frame without adjustment to the size of the shaft line.

60. A trunk supporting exoskeleton for reducing muscle forces in a wearer's back during forward lumbar flexion, the trunk supporting exoskeleton comprising:
a supporting trunk frame configured to be coupled to the wearer's trunk;
a first thigh link configured to be coupled to one of the wearer's thighs, and
a second thigh link configured to be coupled to another of the wearer's thighs, wherein each of the first and second thigh links is rotatably coupled to the supporting trunk frame such that the respective first or second thigh links can flex or extend relative to the supporting trunk frame;
an actuator coupled to the supporting trunk frame, wherein the actuator comprises an actuator housing and an actuator shaft, wherein the actuator is free to rotate relative to the supporting trunk frame, and wherein the actuator shaft is coupled to the first thigh link and the actuator housing is coupled to the second thigh link,
wherein when the wearer bends forward in a sagittal plane, the actuator generates an actuator resistive torque between the actuator housing and the actuator shaft, thereby generating extension torques between the respective first and second thigh links and the supporting trunk frame.

61. The trunk supporting exoskeleton of claim 60, further comprising:
a shaft pulley coupled to the actuator shaft;
a housing pulley coupled to the actuator housing;
a shaft line having a first end wound onto the shaft pulley and a second end coupled to the first thigh link; and
a housing line having a first end wound onto the housing pulley and a second end coupled to the second thigh link,
wherein the actuator resistive torque generates tensile forces in the housing line and the shaft line, thereby generating extension torques between the respective first and second thigh links and the supporting trunk frame.
